(19) United States
(12) Patent Application Publication
(10) Pub. No.: US 2013/0296955 A1
(43) Pub. Date: Nov. 7, 2013

(54) SUTURE DEVICE

(71) Applicants: Charles Haggerty, Monument, CO (US); Kevin Borchard, Colorado Springs, CO (US)

(72) Inventors: Charles Haggerty, Monument, CO (US); Kevin Borchard, Colorado Springs, CO (US)

(21) Appl. No.: 13/886,117

(22) Filed: May 2, 2013

Related U.S. Application Data
(60) Provisional application No. 61/641,469, filed on May 2, 2012.

(51) Int. Cl. A61B 17/04; A61B 17/88

(52) U.S. Cl. CPC A61B 17/0469; A61B 17/88; A61B 17/0401
USPC 606/86 R; 606/144

(57) ABSTRACT

Aspects of the present invention relate to arthroscopic repair surgery and more specifically to rotator cuff tendon tears in the shoulder. One aspect of the invention may comprise a device used to place one or more sutures within the tendon in a grasping type stitch. The suture may then be secured to the humerus bone via a bone anchor or tunnel allowing the tendon to heal.

### PRIORITY

This application claims priority to U.S. Provisional Application No. 6/641,469, filed May 2, 2012, entitled Suture Device for Rotator Cuff Repair and Other Orthopedic Procedures, which is incorporated herein by reference in its entirety.

### FIELD OF THE INVENTION

The present invention relates to medical devices. In particular, but not by way of limitation, the present invention relates to an arthroscopic rotator cuff tendon repair device.

### BACKGROUND OF THE INVENTION

Tendon injuries throughout the body are commonly seen in an orthopedic surgery practice. In general, complete disruption of the bone insertion of a tendon or in the mid-substance of a tendon requires surgical treatment. Repair of the tendon again relies on fixation to bone or to tendon using suture and/or bone tunnels/suture anchors.

Current rotator cuff repair methods and tools rely on an instrument that places a suture through the tendon in a single pass. One technique to place the suture comprises a “shuttle relay” technique which involves using a hollow-bore needle to place a smooth suture through the tendon. This suture is called the ‘passing suture.’ The permanent suture is then tied to the passing suture and is used to pull the permanent suture into place. There are also several known devices that use a harpoon styled device to push the suture through the tendon. In these devices, the permanent suture is loaded on to the device which then grabs the tendon and holds it. The harpoon is then deployed and pierces the permanent suture and pushes it through the tendon. In another method, the tendon may be pierced with a device that has a sharp, tapered end and a grasping jaw. Once the grasping jaw has been passed through the tendon, it can grasp the permanent suture. The suture is then pulled through the tendon.

These current arthroscopic techniques allow the surgeon to only perform a single pass of suture through the tendon at a time. The fixation of the tendon to the bone needs to be secure and last long enough for the tendon to heal, approximately 6-8 weeks. In these devices and techniques, the fixation strength relies on the interface between the suture anchor and bone, the suture and suture anchor, the strength of the suture, and the interface between the suture and tendon.

During a traditional rotator cuff repair, a single, medial row of suture anchors is used to establish contact between the suture and bone. The suture is then passed through the tendon using one of the above known techniques. Additionally, a second, lateral row of suture anchors may be used to increase the bone/tendon/suture interface and increase the contact area between the tendon and bone, the so-called “footprint.” The addition of a second lateral row is known as a double row technique. In both cases, the interface between suture and tendon is the same. A simple stitch or horizontal mattress suture is commonly used.

The healing rate of rotator cuff tendon tears treated in this manner is known to be poor. There is no difference when comparing healing rates between a single or double-row technique. In both cases, the failures tend to be at the suture-tendon interface and come as little surprise. Due to the tight spaces within the shoulder and other joints, it is technically challenging and time consuming to perform a grasping-type suture technique arthroscopically, which is why rotator cuff tendon repairs continue to rely on simple suture techniques.

### SUMMARY OF THE INVENTION

Although improvements to the above-described devices and techniques have focused on improving the strength of the suture anchor/bone interface and the strength of the suture, improving the strength of the suture tendon interface has been neglected in prior devices and techniques. Although physicians generally recognize multiple techniques to place this suture, current techniques in arthroscopic and mini-open repair of rotator cuff tendon do not use grasping type suturing techniques. What is needed is a new suture device and technique that allows a grasping type suture technique to be applied arthroscopically or with mini-open techniques.

As such, one embodiment of the invention comprises medical device comprising a grasping portion, an extension portion, and a spiral-shaped piercing device. The extension portion is one of integrated and coupled to the grasping portion and the spiral-shaped piercing device one of encircles at least a portion of one of the grasping portion and the extension portion, and is encircled by at least one of a portion of the grasping portion and extension portion.

Another embodiment of the invention comprises a method of surgically suturing internal body tissue. One such method comprises creating a surgical endoscopic incision and placing a suture device into the incision, with the suture device comprises a grasping portion. The grasping portion is then extended towards the internal body tissue and moved from a substantially open position to a substantially closed position. In closing the grasping portion, the internal body tissue is grasped by the grasping potion and a spiraled needle is rotated, suturing the body tissue. The spiraled needle is then retracted and the body tissue is released from the grasping portion.

And yet another embodiment of the invention comprises an arthroscopic rotator cuff tendon grasping-type suturing system comprising a pair of actuation triggers. A first of the actuation triggers is operatively coupled to a pair of jaws and a second of the actuation triggers is operatively coupled to a cannulated needle. The cannulated needle comprises one or more sutures.

The above-described embodiments and implementations are for illustration purposes only. Numerous other embodiments, implementations, and details of the invention are easily recognized by those of skill in the art from the following descriptions and claims.

### BRIEF DESCRIPTION OF THE DRAWINGS

Various objects, advantages and an understanding of the present invention are more readily appreciated by reference to the following Detailed Description and to the appended claims when taken into account with the accompanying Drawings, wherein:

FIG. 1 illustrates a side view of a portion of a medical device including an grasping portion and a corkscrew shaped suture needle constructed in accordance with aspects of the present invention;

FIG. 2 illustrates a side view of the corkscrew shaped suture needle of FIG. 1 constructed in accordance with aspects of the present invention;

FIG. 3 illustrates a side view of a device where body tissue is located between a pair of grasping portion jaws in a closed position and a suture needle has made multiple passes through the tissue being sutured;

FIG. 4 illustrates a side view of a device comprising a housing and an actuation device used to deploy and operate the suture system constructed in accordance with aspects of the present invention;

FIG. 5 illustrates four sequential images of a portion of a device being used to deploy the suture needle, insert the suture through multiple passes of the needle into the tissue, retract the needle, and retract the deployment element, leaving the suture in the body tissue, according to one embodiment of the invention;

FIG. 6 illustrates a side view of another embodiment of a device constructed in accordance with aspects of the present invention;

FIG. 7 illustrates a method that may be carried out with the embodiments described herein;

FIG. 8A illustrates a top view of a grasping portion and corkscrew shaped suture needle of a medical device constructed in accordance with aspects of the present invention;

FIG. 8B illustrates a side view of a grasping portion and corkscrew shaped suture needle of a medical device constructed in accordance with aspects of the present invention; and

FIG. 8C illustrates an end view of a grasping portion and corkscrew shaped suture needle of a medical device constructed in accordance with aspects of the present invention.

### DETAILED DESCRIPTION

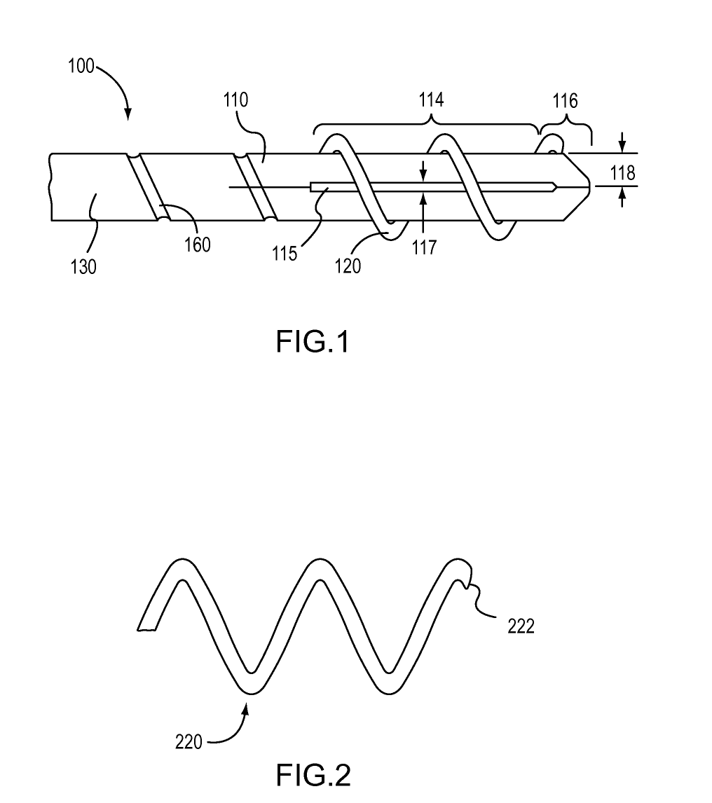

Turning first to FIG. 1, seen is one embodiment of a portion of a medical device 100. The portion of the device 100 seen in FIG. 1 comprises a grasping portion 110 and a spiral-shaped piercing device 120. As seen in FIG. 1, the grasping device 110 may comprise a pair of jaws and the spiral-shaped piercing device 120 may comprise a corkscrew-shaped needle. The jaws may be substantially parallel and may be longitudinally-aligned. Furthermore, the jaws may be referred to herein as jaw sections. The use of the grasping portion 110 provides the ability to implement a grasping-type suture technique, which may prevent a placed suture from sliding along longitudinal-aligned collagen fibers in the sutured body tissue.

The grasping portion 110 may be one of integrated to and coupled to an extension portion 130. The term integrate, integrated, and other similar terms may be used throughout the specification to describe a singular entity or unit while the term couple, coupled, and other similar terms may refer to individual parts that have been joined together to form a larger unit, where appropriate.

Turning briefly to FIG. 4, seen is a device 400 comprising a housing 440 and a trigger 450 where a top jaw section 412 is hingedly and/or rotatably coupled to the grasping portion 420. The top jaw section 412 may also be hingedly and/or rotatably coupled to the extension portion 430. Furthermore, as seen in FIG. 4, and in returning to FIG. 1, the spiral-shaped piercing device 120, 420 which may comprise an elongated spiral-shaped needle, may encircle, or be adapted to encircle, at least a portion of the grasping portion 110, 410.

As seen in FIG. 1, the jaw sections may comprise a middle portion 114 and a distal end 116. When the grasping portion is in a closed position, as seen in FIG. 1, there may be an open space 115 between the jaw sections in at least part of the middle portion 114. Additionally, when in the closed position, at least a portion of the distal end 116 of a jaw section may be in contact with at least a portion of the distal end 116 of the other jaw section. Furthermore, it is contemplated that a height 118 of each jaw section may comprise about 5 mm A width of each jaw section may also comprise about 5 mm Each of the width and height 118 may fall within the range of about 4 mm to about 6 mm, although alternative sizes are contemplated. A length of each jaw section may comprise about 20 mm, although the length of teach jaw section may comprise about 15-25 mm in one embodiment. The open space distance 117 may comprise a distance of about 0.5 mm, although the range of the distance 117 may be from about 0.1 mm to about 1 mm However, larger or smaller (pediatric) sizes are contemplated. Furthermore, generally circular jaw sections and extension portions 130 are contemplated.

As seen in FIG. 1, at least part of the extension portion 130 and/or the grasping portion 110 may comprise one or more grooves, such as, but not limited to, the spiraled groove 160 seen in FIG. 1. This groove 160 may extend along at least a portion of the length of the extension portion 130 and/or grasping portion 110 and may be adapted to receive the spiral-shaped piercing device 120. Alternatively, or additionally, the groove 160 may be located on an inner surface of the housing 440.

Turning now to FIG. 2, seen is the spiral-shaped piercing device 220. One spiral-shaped piercing device 220 may comprise a cannulated needle. For example, a tip 222 of the cannulated needle may be adapted to pierce body tissue such as, but not limited to, a rotator cuff tendon. The needle may comprise a threadable and flexible suture material such as, but not limited to, a polymeric thread, and the suture material may be secured to the needle (i.e. by an anchor, a notch in the needle, or a knot). It is contemplated that the suture material may be removed from the needle—for example, by retracting the needle when the suture material is coupled to the needle through a notch, or through the use of a trigger 450 as seen in FIG. 4.

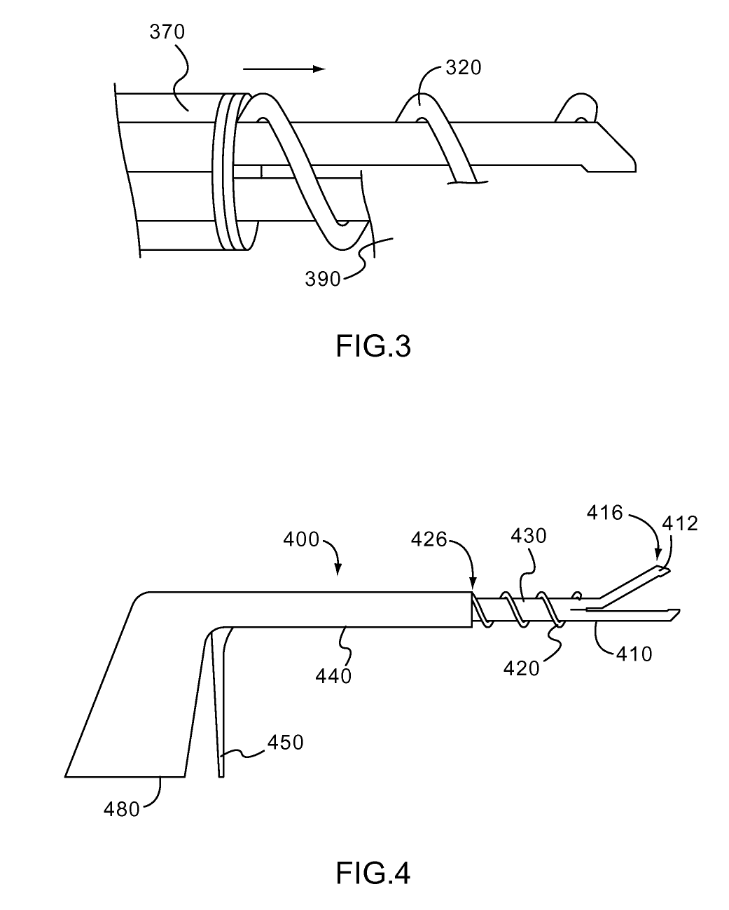

The trigger 450 may comprise one or more triggers, with each trigger being operatively coupled to one or more of the grasping portion 410, extension portion 430 and/or the spiral-shaped piercing device 420. As seen in FIG. 3, in one embodiment, the spiral-shaped piercing device 320 may be one of coupled and integrated to a bearing platform 370. One of the triggers 450 may be coupled and/or integrated to the bearing platform 370 and may move the bearing platform 370 towards the tissue 390. The spiral-shaped piercing device 320 may then be rotated through the tissue 390 upon reaching the tissue 390. Movement of the trigger 450 towards or away from the handle 480 may move (i) the grasping portion between the closed position seen in FIG. 1 and the open position seen in FIG. 4, and vice versa, (ii) the spiral-shaped piercing device 420 from the semi-retracted position seen in FIG. 4 to the extended position seen in FIG. 1, and vice versa, and (iii) the grasping portion distal end 116 from a position proximal a housing distal end 426 to the position seen in FIG. 4, and vice versa. It is contemplated that other open/closed grasping positions and retracted and extended positions beyond those seen in the figures are contemplated.

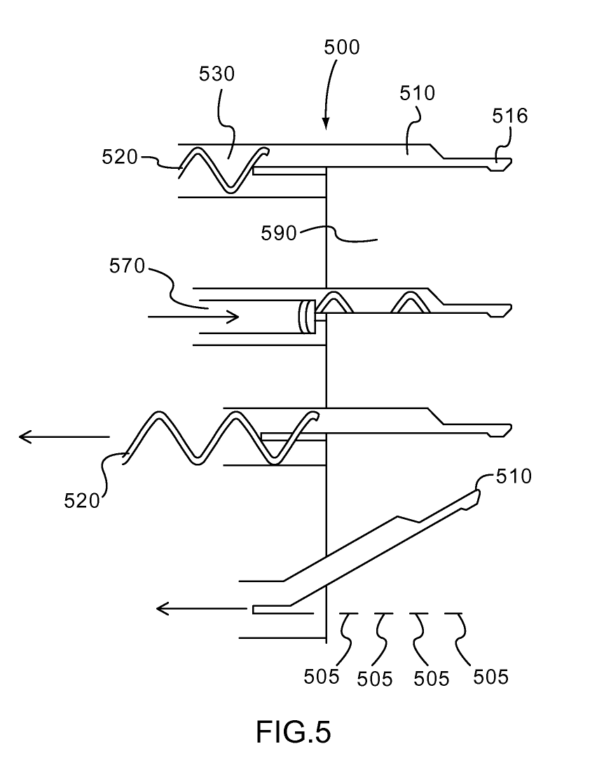

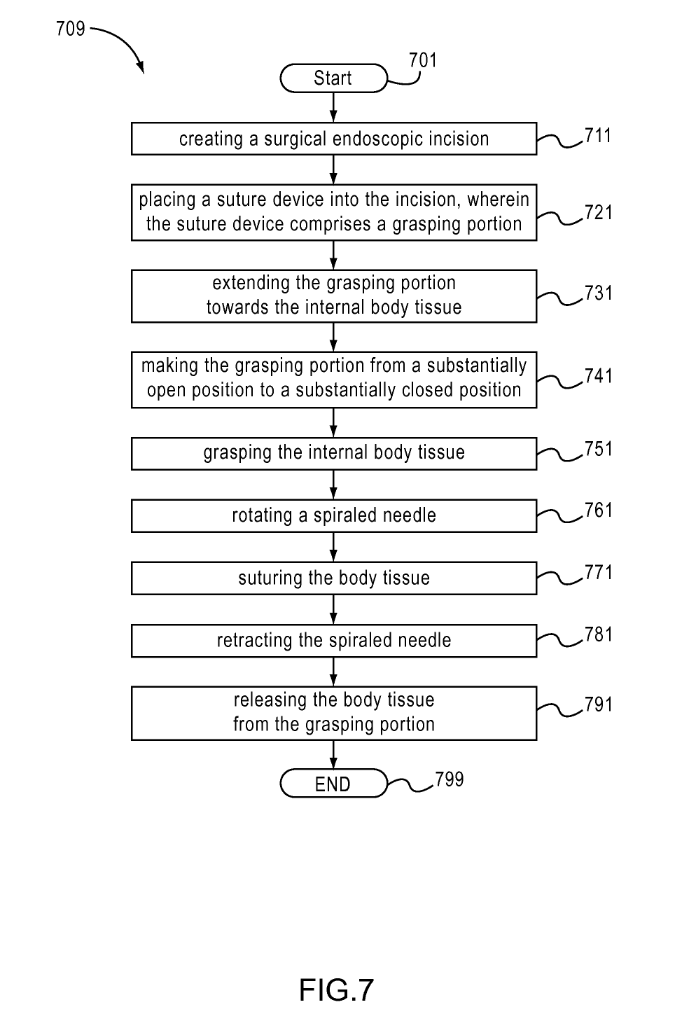

Turning now to FIG. 7, seen is one method 709 of surgically suturing internal body tissue. One method starts at 701 and at 711 the method 709 comprises creating a surgical endoscopic incision. One incision may be made in the shoulder. At 721 the method comprises placing a suture device such as the device seen in FIGS. 1-6, into the incision. One such device comprises the grasping portion 110 seen in FIG. 1. At step 731, the method 709 comprises extending the grasping portion 110 toward the internal body tissue such as, but not limited to, a rotator cuff tendon, and at 741 the method 709 comprises moving the grasping portion from a substantially open position to a substantially closed position. The method 709 at 751 comprises grasping the internal body tissue. Turning now to FIG. 5, seen is a grasping portion 510 in a substantially closed position with a body tissue 590 between the distal ends 516. The embodiment seen in FIG. 5 may comprise a substantially hollow extension portion 530 and grasping portion 510, allowing the spiral-shaped piercing device 520 to be placed inside the extension portion 530 and grasping portion 510. As the body tissue 590 is grasped and held in place by the grasping portion 510, the spiral-shaped piercing device 520 may be moved towards the tissue 590 with the bearing platform 570, and the needle may be deployed internally within the device 500. Although FIG. 5 shows the spiral-shaped piercing device 520 and bearing platform 570 as internal to and encompassed by the device 500, it is contemplated that the spiral-shaped piercing device 520 and bearing platform 570 may also encompass the extension portion 530 and grasping portion 510 in a working embodiment, as seen in FIG. 1. In the FIG. 5 embodiment, the extension portion 530 and grasping portion 510 may comprise the grooves 160 seen in FIG. 1, located on an internal extension portion 530 and/or grasping portion 510 surface. In the FIG. 5 embodiment, as with the FIG. 1 embodiment, the suture may be contained within the cannulated needle.

Upon grasping the internal body tissue 590, and as seen at steps 761 and 771 of the method 709, the spiral-shaped piercing device 520 comprising a spiraled needle may be rotated and the suture may be placed in the tissue. Then, and as seen at steps 781 and 791, the spiraled needle may be retracted and the body tissue 590 may be released from the grasping portion 510 and the device 500 may be removed, leaving only the suture 505 in the tissue. This increases the strength of the suture-tendon interface and significantly reduces the risk of failure. Healing rates for tendon repairs using a grasping type suture technique are consistently high.

Although the embodiment shown in FIG. 5 is described with reference to the method of FIG. 7, the embodiment seen in FIG. 1 and elsewhere may also be used. For example, the spiraled needle may be rotated around the grasping portion 110, as seen in FIG. 1. Rotating a spiraled needle around the grasping portion may comprise advancing the spiraled needle from a retracted starting position, as seen in, for example, FIG. 4 and the first image of FIG. 5, to an extended ending position after the grasping portion is in the substantially closed position, as seen in, for example, FIGS. 1, 3, and the second image in FIG. 5.

Moving the grasping portion 110 from a substantially open position to a substantially closed position and grasping the internal body tissue 590 may comprise engaging a first trigger 450 on the device 400, which may be referred to as a suture device. Furthermore, rotating a spiraled needle around the grasping portion, suturing the body tissue, and retracting the spiraled needle comprises engaging a second trigger 450 on the suture device 400. Additionally, grasping the internal body tissue comprises stabilizing the internal body tissue in a substantially fixed position. It is further contemplated that the steps of rotating a spiraled needle of 761, suturing the body tissue of 771, and retracting the spiraled needle 781 may be repeated. One method 709 may further comprise creating a grasping stitch.

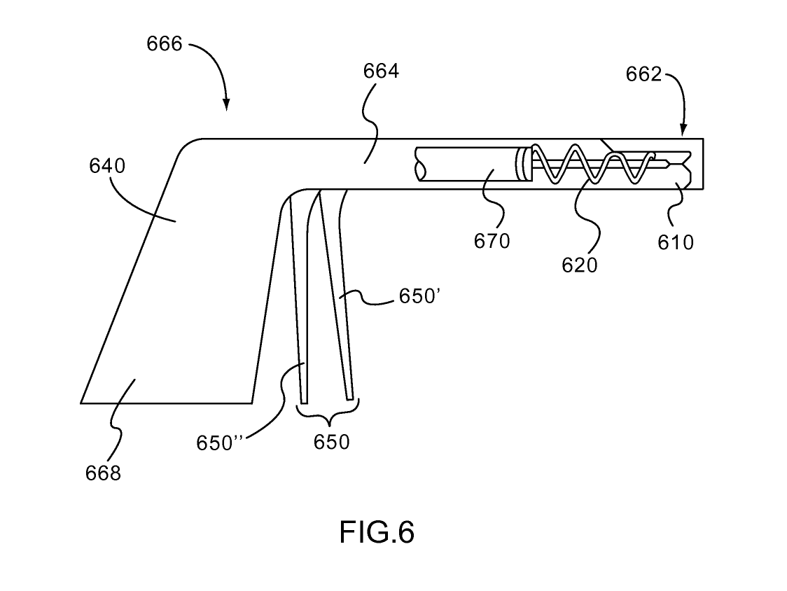

Turning now to FIG. 6, seen is an arthroscopic grasping-type suturing system 666. One such system 666 may be similar to the device 100 seen in reference to FIG. 1 and elsewhere. The system 666 in FIG. 6 comprises a pair of actuation triggers 650. A first of the actuation triggers may operatively coupled to a pair of jaws comprising the grasping portion 610. A second of the actuation triggers may be operatively coupled to a spiral-shaped piercing device 620 comprising a cannulated needle. The cannulated needle may include one or more suture threads. The cannulated needle 620 is in a retracted position in FIG. 6, ready to be extended and suture the tissue.

Operation of a first trigger 650′ may deploy the jaws from the retracted position of GI. 6 to a forward position. The first trigger 650′ may also change the jaws between an open and a closed position and may hold a rotator cuff tendon in a substantially stable position when the jaws are in the closed position. Operation of a second trigger 650″ may deploy the cannulated needle along a longitudinal axis of the jaws when the jaws are in an extended and closed position. The operation of the second trigger 650″ may further deploy the cannulated needle in a corkscrew motion along the grooves which may be located in a system housing 640. The operation of the second trigger may further suture the rotator cuff tendon, remove the suture from the cannulated needle, and retract the cannulated needle from the suture, leaving the placed suture in the tissue. The cannulated needle may be located along a longitudinal axis to the jaws when the jaws are in the closed position. Additionally, it is contemplated that one or more sutures may be coupled to a humerus or other bone via at least one of one or more bone anchors and tunnels.

The FIG. 6 system may comprise a pistol grip type handle 668 with a narrow, elongated end 664. The elongated end 664 may be adapted for introduction through small incisions into the body, such as, but not limited to, a shoulder location. At its end 662 are grasping jaws that can grab and hold the tendon. The corkscrew cannulated needle may be wound within the elongated distal end of the system 666, mounted to the bearing platform 670.

In one embodiment, with the tendon held in place by the first trigger 650′, the second trigger 650″ may deploy the cannulated needle down the length of the instrument over, or through, the grasping jaws. Grooves may guide the needle into a corkscrew motion, rotating around the instrument as it advances forward.

The cannulated needle, which is shaped like a corkscrew and can be advanced in a corkscrew-type motion, is an improvement over the older methods because it allows the surgeon to place multiple suture passes through the tendon at one time, using a simple motion, which creates the preferred grasping-type stitch. This provides the user with a stronger suture/tendon interface. Because most rotator cuff tendon repairs fail at the suture/tendon interface, the invention will improve the healing rates of these repairs.

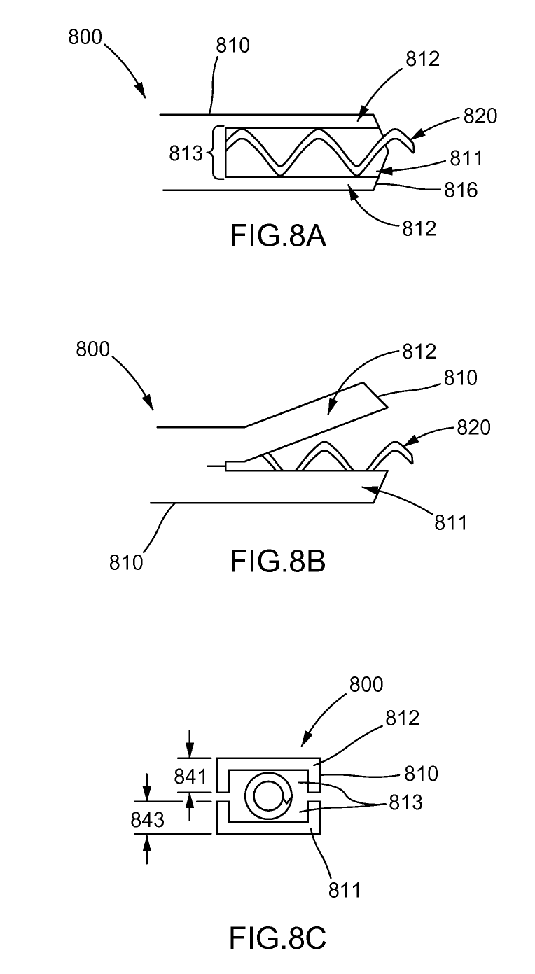

Turning now to FIGS. 8A, 8B, and 8C, seen is a grasping portion 810 of another embodiment of a medical device 800. As seen in FIGS. 8A and 8B, at least part 813 of at least the top jaw section 812 of grasping portion 810 may be removed. Removal of the part 813 of the top jaw section 812 may enable or otherwise facilitate passage of the spiral-shaped piercing device 820 through the grasping portion 810 by providing a passageway for the spiral-shaped piercing device 820 to travel as it moves towards the distal end 816 of the device 800 to suture the tissue in the device jaws when the jaws are in a closed position. Such an embodiment, and other embodiments, may be used to suture tissue when the jaws are in the open or closed position. As also seen in FIG. 8C, it is contemplated that the part 813 of the top jaw section 812 and/or the lower jaw section 811 that has been removed may not comprise the entire height 841, 843 of the top jaw section 812 or lower jaw section 811 and may only comprises a portion of the height.

Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

What is claimed is:

1. A medical device comprising,
a grasping portion;
an extension portion, the extension portion being one of integrated and coupled to the grasping portion; and
a spiral-shaped piercing device one of,
encircling at least a portion of one of the grasping portion and the extension portion, and
is encircled by at least a portion of one of the grasping portion and the extension portion.

2. The medical device of claim 1 wherein,
the grasping portion comprises a pair of substantially parallel and longitudinally-aligned jaw sections; and
at least one of the jaw sections is rotatably coupled to the at least one of the extension portion, grasping portion, and a device housing.

3. The medical device of claim 2 wherein,
each of the jaw sections comprise a distal end and a middle portion; and
when the grasping portion is in a closed position,
there is an open space between the middle portion of the jaw sections, and
at least a portion of the distal end of each jaw section is in contact with at least a portion of the distal end of the other jaw section.

4. The medical device of claim 3 wherein,
the jaw sections comprise a height of about 5 mm; and
the open space comprises a distance of about 0.5 mm

5. The medical device of claim 1 further comprising,
a housing; wherein,
at least one of the housing, grasping portion and extension portion comprise at least one groove; and
the spiral-shaped piercing device comprises a cannulated needle placed within at least a portion of the at least one groove.

6. The medical device of claim 5 wherein, the cannulated needle comprises a threadable suture material.

7. The medical device of claim 5 further comprising an actuator portion operatively coupled to at least one of the grasping portion, extension portion, and piercing device, wherein,
the actuator portion comprises one or more trigger mechanisms; and
at least one of the one or more trigger mechanisms,
extends the grasping portion between a retracted position and an extended position,
adjusts the grasping portion between an open position and a closed position, and
advances and retracts the cannulated needle.

8. The medical device of claim 1, wherein, the spiral-shaped piercing device comprises a corkscrew shape.

9. A method of surgically suturing internal body tissue comprising,
creating a surgical endoscopic incision;
placing a suture device within the incision, wherein the suture device comprises a grasping portion;
extending the grasping portion towards the internal body tissue;
moving the grasping portion from a substantially open position to a substantially closed position;
grasping the internal body tissue;
rotating a spiraled needle;
suturing the body tissue;
retracting the spiraled needle; and
releasing the body tissue from the grasping portion.

10. The method of claim 9 wherein,
the incision is created in the shoulder;
the body tissue comprises a rotator cuff tendon; and
the spiraled needle is one of,
rotated around the grasping portion, and
rotated within the grasping portion.

11. The method of claim 9 wherein, rotating a spiraled needle comprises advancing the spiraled needle from a retracted starting position to an extended ending position after the grasping portion is in the substantially closed position.

12. The method of claim 9 wherein,
moving the grasping portion from a substantially open position to a substantially closed position and grasping the internal body tissue comprises engaging a first trigger on the suture device; and
rotating a spiraled needle around the grasping portion, suturing the body tissue, and retracting the spiraled needle comprises engaging a second trigger on the suture device.

13. The method of claim 9 wherein, rotating a spiraled needle comprises rotating the spiraled needle through one or more grooves.

14. The method of claim 9 wherein, grasping the internal body tissue comprises stabilizing the internal body tissue in a substantially fixed position.

15. The method of claim 9, further comprising,
repeating the steps of rotating a spiraled needle, suturing the body tissue, and retracting the spiraled needle in order to place multiple suture passes through the body tissue; and
creating a grasping stitch.

16. An arthroscopic rotator cuff tendon grasping-type suturing system comprising,
a pair of actuation triggers, wherein,
a first of the actuation triggers is operatively coupled to a pair of jaws, and
a second of the actuation triggers is operatively coupled to a cannulated needle, wherein the cannulated needle comprises one or more sutures.

17. The system of claim 16 wherein,
operation of the first trigger changes the jaws between an open and a closed position, holding a rotator cuff tendon in a substantially stable position; and
operation of the second trigger deploys the cannulated needle along a longitudinal axis of the jaws when the jaws are in the closed position.

18. The system of claim 17 wherein, the operation of the second trigger further deploys the cannulated needle in a corkscrew motion along internal grooves in a system housing.

19. The system of claim 17 wherein, the operation of the second trigger further,
sutures the rotator cuff tendon;
removes the suture from the cannulated needle; and
retracts the cannulated needle from the suture.

20. The system of claim 17 wherein,
the cannulated needle is along a longitudinal internal to the jaws when the jaws are in the closed position; and
the one or more sutures being coupled to a humerus bone via at least one of one or more bone anchors and tunnels.
